(19) United States
(12) Patent Application Publication
(10) Pub. No.: US 2018/0339136 A1
(43) Pub. Date: Nov. 29, 2018

(54) CATHETER GRIP DEVICE AND METHOD

(71) Applicant: RA MEDICAL SYSTEMS, INC., Carlsbad, CA (US)

(72) Inventors: Zachary WOOD, Encinitas, CA (US); Travis KOEHN, Murrieta, CA (US); Dean IRWIN, Carlsbad, CA (US)

(21) Appl. No.: 15/987,838

(22) Filed: May 23, 2018

Related U.S. Application Data
(60) Provisional application No. 62/510,953, filed on May 25, 2017.

(51) Int. Cl. A61M 25/02

(52) U.S. Cl. CPC A61M 25/02; A61B 18/24

(57) ABSTRACT

A catheter grip is disclosed that may be engaged onto a catheter by squeezing respective separation regions of the catheter grip to open a gap between distal ends of respective rigid plates thereof. The catheter grip may then be used to grip and manipulate the catheter by releasing the force on the separation regions and squeezing respective compression regions of the respective rigid plates of the catheter grip.

### RELATED APPLICATIONS

This application claims priority from U.S. Provisional Patent Application Ser. No. 62/510,953, filed on May 25, 2017, by Z. Wood et al. titled “Catheter Grip Device and Method”, which is incorporated by reference herein in its entirety.

### BACKGROUND

Medical catheter procedures may include angioplasty, angiograms, electrophysiology procedures and the like. Catheters often pass through tortuous paths, and torque and axial force may need to be applied to the medical catheter to deliver to a target and perform a desired procedure. Additionally the medical catheter may be inserted into a patient's body through a hemostatic introducer, sheath, or guide catheter, and this can result in relative friction between the introducer, sheath, or guide catheter and the medical catheter and therefore increase the force required to advance the medical catheter to a target site. Many medical catheters include fragile components such as fiber optics, electrical components or the like. When a user of the medical catheter is performing a procedure, the user may deform the shaft of the medical catheter with finger pressure or the like which may be unevenly applied and which may lead to reduced performance of the medical catheter. What have been needed are devices and methods which allow a user of a medical catheter to advance, retract, and apply torque to a medical catheter without deforming the medical catheter shaft or components disposed within a shaft of the medical catheter.

### SUMMARY

Some embodiments of a catheter grip may include a first rigid plate that has a proximal end, a distal end, a compression region on an outside surface of the first rigid plate, and a separation region on the outside surface proximal of the compression region. The first rigid plate may also include a first elongate slot which has a cylindrical contour and which extends transversely across an inside surface of the distal end of the first rigid plate. The first elongate slot may also include a first longitudinal axis of curvature.

The catheter grip may also include a second rigid plate which has a proximal end, a distal end, a compression region on an outside surface of the second rigid plate, and a separation region on the outside surface proximal of the compression region. The second rigid plate may also include a second elongate slot which has a cylindrical contour and which extends transversely across an inside surface of the distal end of the second rigid plate. The second elongate slot may have a second longitudinal axis of curvature and may be disposed such that it is opposite to and in alignment with the first elongate slot.

The catheter grip may also include a resilient rib which pivotally couples the first rigid plate to the second rigid plate such that the respective longitudinal axes of the first elongate slot and the second elongate slot remain aligned during pivoting of the first rigid plate relative to the second rigid plate. The resilient rib may extend from the inside surface of the first rigid plate to the inside surface of the second rigid plate. The resilient rib allows the first rigid plate and the second rigid plate to be displaced in a relative pivoting motion along a pivot axis which is parallel to the respective longitudinal axes of the first and second elongate slots. The resilient rib may be disposed between the respective separation region and compression region of each of the first and second rigid plates.

Some embodiments of a catheter grip may include a first rigid plate which has a proximal end, a distal end, and a compression region on an outside surface of the first rigid plate. The first rigid plate may also have a separation region which may be disposed on the outside surface of the first rigid plate proximal to the compression region. The first rigid plate may also include a first elongate slot, the surface of which may be formed as a cylindrical contour. The first elongate slot can extend transversely across an inside surface of the distal end of the first rigid plate. The first elongate slot may also include a first longitudinal axis of curvature which is located at a centroid of the cylindrical contour.

The catheter grip may also include a second rigid plate which has a proximal end, a distal end, and a compression region on an outside surface of the second rigid plate. The second rigid plate may also have a separation region which may be disposed on the outside surface of the second rigid plate proximal to the compression region. The second rigid plate may also include a second elongate slot, the surface of which may be formed as a cylindrical contour. The second elongate slot may extend transversely across an inside surface of the distal end of the second rigid plate. The second elongate slot may also include a second longitudinal axis of curvature which is located at a centroid of the cylindrical contour. The second elongate slot may be disposed such that it is opposite to and in alignment with the first elongate slot.

The catheter grip may further include a resilient rib which pivotally couples the first rigid plate to the second rigid plate such that respective longitudinal axes of the first elongate slot and second elongate slot will remain substantially aligned during the pivoting of the first rigid plate relative to the second rigid plate. For some catheter grip embodiments, the resilient rib may extend from the inside surface of the first rigid plate to the inside surface of the second rigid plate, thus allowing for the first rigid plate and second rigid plate to be displaced in a relative pivoting motion along a pivot axis which is disposed such that it is parallel to the respective longitudinal axes of the first and second elongate slots. The resilient rib may also be disposed such that it is located between the respective separation region and compression region of each of the first and second rigid plates.

In some instances when the resilient rib is disposed in a relaxed state, there is a gap between the inside surfaces of the respective proximal ends and distal ends of the first and second rigid plates, and the first and second rigid plates are substantially parallel to each other. Additionally, the first and second longitudinal axes of curvature of the respective elongate slots may be aligned so as to define an incomplete cylindrical bore. The incomplete cylindrical bore may have a nominal inside diameter which is slightly larger than a nominal outer diameter of a catheter which may be engaged in the incomplete cylindrical bore. In this manner the first and second elongate slots mechanically capture the outside contour of the catheter, however the catheter grip is free to easily slide along the axial length of the catheter with the resilient rib disposed in the relaxed state.

In some instances, when a separation force is applied to each of the respective separation regions of the first rigid plate and the second rigid plate the resilient rib may be elastically deformed so as to expand the gap between the respective distal ends of the first and second rigid plates via rotation of the first and second rigid plates about the pivot axis until the gap is greater than an outside transverse dimension or diameter of the catheter. When the separation force is applied to each of the respective separation regions with a magnitude of force sufficient to overcome a resilient resistance of the resilient rib so as to displace the first and second rigid plates relative to each other and approximate the respective separation regions towards each other, the catheter may be engaged into or disengaged from the elongate slots with non-damaging elastic deformation of the catheter.

In addition, when a compressive force is applied to each of the respective compression regions of the first rigid plate and the second rigid plate, the resilient rib may be elastically deformed and the first and second rigid plates rotationally displaced so as to reduce the gap between the respective distal ends of the first and second rigid plates via rotation of the first and second rigid plates relative to each other about the pivot axis. The reduction in the gap and approximation of the elongate slots over the catheter allows for gripping of the catheter via compression of the outside surface of the catheter with the respective inside surfaces of the first and second elongate slots.

Some embodiments of a method for gripping and releasing a catheter utilizing a catheter grip may include applying a first separation force to a separation region of first rigid plate of a catheter grip, and applying a second separation force which is opposed to the first separation force to a separation region of a second rigid plate of the catheter grip so as to elastically deform a resilient rib which pivotally couples the first rigid plate to the second rigid plate and to expand a gap between a distal end of the first rigid plate and a distal end of the second rigid plate.

The method for gripping and releasing a catheter utilizing a catheter grip may further include engaging an outside surface of the catheter with an incomplete cylindrical bore formed by a first elongate slot of the first rigid plate and with a second elongate slot of the second rigid plate. The method may further include applying a first compressive force to a compression region of the first rigid plate, and applying a second compressive force which is opposed to the first compressive force to a compression region of the second rigid plate so as to elastically deform the resilient rib and to reduce the gap between the distal ends of the first and second rigid plates. An outside surface of the catheter may thereby be gripped with respective inside surfaces of the first and second elongate slots due to frictional forces between those surfaces. The method for gripping and releasing a catheter utilizing a catheter grip may further include manipulating the catheter with the catheter grip.

Some embodiments of a method for gripping and releasing a catheter utilizing a catheter grip may include providing a catheter grip. The catheter grip may include a first rigid plate having a proximal end, a distal end, and a compression region on an outside surface of the first rigid plate. The catheter grip may also include a separation region on the outside surface which is located proximal of the compression region. The catheter grip may also include a first elongate slot having a cylindrical contour which extends transversely across an inside surface of the distal end of the first rigid plate, with the first elongate slot having a first longitudinal axis of curvature.

The catheter grip may also include a second rigid plate which may have a proximal end, a distal end, a compression region on an outside surface of the second rigid plate. The second rigid plate may also have a separation region on the outside surface of the second rigid plate which is located proximal of the compression region. The second rigid plate may also include a second elongate slot having a cylindrical contour and which extends transversely across an inside surface of the distal end of the second rigid plate. The second elongate slot may include a second longitudinal axis of curvature, and may be disposed opposite to and in alignment with the first elongate slot.

The catheter grip may also include a resilient rib which pivotally couples the first rigid plate to the second rigid plate such that respective longitudinal axes of the first elongate slot and second elongate slot remain aligned during pivoting of the first rigid plate and second rigid plate. The resilient rib may extend from the inside surface of the first rigid plate to the inside surface of the second rigid plate thereby allowing the first rigid plate and second rigid plate to be displaced in a relative pivoting motion along a pivot axis which is parallel to the respective longitudinal axes of the first and second elongate slots. The resilient rib may be disposed between the respective separation region and compression region of each of the first and second rigid plates.

The method for gripping and releasing a catheter utilizing a catheter grip may further include applying a separation force to the respective separation regions so as to expand a gap between the distal ends of the first and second plates until the gap is greater than the outside diameter of the catheter. The method may further include engaging and gripping the catheter with inside surfaces of the elongate slots without deformation of the catheter by inserting the catheter through the gap between the distal ends of the first and second rigid plates.

The method for gripping and releasing a catheter utilizing a catheter grip may further include applying a compressive force to the respective compression regions of the rigid plates so as to elastically deform the rib and to reduce the gap between the distal ends of the first and second plates, thereby engaging the outside surface of the catheter with the respective inside surfaces of the first and second elongate slots.

The method for gripping and releasing a catheter utilizing a catheter grip may further include releasing the compressive force thereby transitioning the resilient rib to a relaxed state wherein, in some cases, the first and second rigid plates are substantially parallel to each other, and the first and second longitudinal axes of curvature of the respective elongate slots are aligned so as to define an incomplete cylindrical bore. The incomplete cylindrical bore may have a nominal inside diameter which is slightly larger than a nominal outer diameter of the catheter engaged in the incomplete cylindrical bore, such that the first and second elongate slots mechanically capture the catheter but may be easily slid along the axial length of the catheter. The method may further include sliding the axial grip along the axial length of the catheter while the resilient rib is in a relaxed state.

Certain embodiments are described further in the following description, examples, claims and drawings. These features of embodiments will become more apparent from the following detailed description when taken in conjunction with the accompanying exemplary drawings.

### BRIEF DESCRIPTION OF THE DRAWINGS

The drawings are intended to illustrate certain exemplary embodiments and are not limiting. For clarity and ease of illustration, the drawings may not be made to scale and, in some instances, various aspects may be shown exaggerated or enlarged to facilitate an understanding of particular embodiments.

FIG. 1 is a perspective view of a catheter grip embodiment including a first rigid plate having a first elongate slot, a second rigid plate having a second elongate slot, and a resilient rib which pivotally couples the first rigid plate to the second rigid plate.

FIG. 2 is an elevation view of the catheter grip embodiment of FIG. 1, the first rigid plate including a separation region and a compression region and the second rigid plate including a separation region and a compression region.

FIG. 3 is an elevation view of the catheter grip embodiment of FIG. 1 depicting an inside surface of the distal end of the first rigid plate, an inside surface of the distal end of the second rigid plate, and a gap disposed between the inside surfaces of the distal ends of the first and second rigid plates.

FIG. 4 is perspective view of the catheter grip embodiment of FIG. 1 with a separation force applied to each of the respective separation regions of the first rigid plate and the second rigid plate, the application of the separation force resulting in expansion of the gap between the inside surfaces of the distal ends of the first and second rigid plates.

FIG. 5 is an elevation view of the catheter grip embodiment of FIG. 1 with a separation force applied to each of the respective separation regions of the first rigid plate and the second rigid plate, with the application of the separation force resulting in expansion of the gap between the inside surfaces of the distal ends of the first and second rigid plates.

FIG. 6 is an elevation view of the catheter grip embodiment of FIG. 1 with a separation force applied to each of the respective separation regions of the first rigid plate and the second rigid plate, with the application of the separation force resulting in expansion of the gap between the inside surfaces of the distal ends of the first and second rigid plates.

FIG. 7 is a perspective view of the catheter grip embodiment of FIG. 1 with a compression force applied to each of the respective compression regions of the first rigid plate and the second rigid plate, with the application of the compression force resulting in reduction of the gap between the inside surfaces of the distal ends of the first and second rigid plates.

FIG. 8 is an elevation view of the catheter grip embodiment of FIG. 1 with a compression force applied to each of the respective compression regions of the first rigid plate and the second rigid plate, with the application of the compression force resulting in reduction of the gap between the inside surfaces of the distal ends of the first and second rigid plates.

FIG. 9 is an elevation view of the catheter grip embodiment of FIG. 1 with a compression force applied to each of the respective compression regions of the first rigid plate and the second rigid plate, with the application of the compression force resulting in reduction of the gap between the inside surfaces of the distal ends of the first and second rigid plates.

FIG. 10 is an elevation view of a patient undergoing a catheter procedure utilizing a laser and a laser ablation catheter while being imaged with a fluoroscope.

FIG. 11 is a perspective view of a guide catheter embodiment and the surface of tissue of the patient of FIG. 10, the guide catheter embodiment being inserted into the tissue and into a suitable arterial or venous lumen of the patient.

FIG. 12 is a perspective view of the guide catheter embodiment and patient tissue each of FIG. 11 after insertion of a laser ablation catheter embodiment into the introducer, sheath, or guide catheter embodiment.

FIG. 13 is a perspective view of the guide catheter embodiment, patient tissue, and laser ablation catheter shown in FIG. 12 with the catheter grip embodiment of FIG. 1 disposed in proximity to the laser ablation catheter, and the resilient rib of the catheter grip embodiment disposed in a relaxed state.

FIG. 14 is a perspective view of the guide catheter embodiment, patient tissue, laser ablation catheter, and catheter grip embodiment shown in FIG. 13 with a separation force applied to each of the respective separation regions of the first and second rigid plates of the catheter grip embodiment resulting in expansion of the gap between the inside surfaces of the distal ends of the first and second rigid plates.

FIG. 15 is an elevation view of the laser ablation catheter and catheter grip embodiment shown in FIG. 14 with a separation force applied to each of the respective separation regions of the first and second rigid plates of the catheter grip embodiment so as to expand the gap between the inside surfaces of the distal ends of the first and second rigid plates.

FIG. 16 is a perspective view of the guide catheter embodiment, patient tissue, laser ablation catheter, and catheter grip embodiment shown in FIG. 13 with the resilient rib of the catheter grip embodiment disposed in a relaxed state and the catheter mechanically captured between the first and second elongate slots such that the catheter grip may be easily slid along the axial length of the catheter.

FIG. 17 is an elevation view of the laser ablation catheter and catheter grip embodiment shown in FIG. 16 with the resilient rib of the catheter grip embodiment disposed in a relaxed state and the catheter mechanically captured within the first and second elongate slots such that the catheter grip may be easily slid along the axial length of the catheter.

FIG. 18 is a perspective view of the guide catheter embodiment, patient tissue, laser ablation catheter, and catheter grip embodiment shown in FIG. 13 with a compression force applied to each of the respective compression regions of the catheter grip so as to elastically deform the rib such that the gap between the first and second rigid plates is reduced and an outside surface of the catheter is compressed with the respective inside surfaces of the first and second elongate slots.

FIG. 19 is an elevation view of the guide catheter embodiment, laser ablation catheter, and catheter grip embodiment shown in FIG. 13 with a compression force applied to the respective compression regions of the catheter grip so as to elastically deform the rib such that the gap between the distal ends of the first and second rigid plates is reduced and an outside surface of the catheter is compressed with inside surfaces of the first and second elongate slots.

FIG. 20 is an elevation view in partial section showing fingers of a user deforming the shaft of a laser ablation catheter.

FIG. 21 is a perspective view of the guide catheter embodiment, patient tissue, laser ablation catheter, and catheter grip embodiment shown in FIG. 13, with the catheter grip engaged with and gripping an outside surface of the laser ablation catheter and the catheter grip advancing the laser ablation catheter into the guide catheter embodiment.

FIG. 22 is a perspective view of the guide catheter embodiment, patient tissue, laser ablation catheter, and catheter grip embodiment shown in FIG. 13 with the resilient rib of the catheter grip embodiment disposed in a relaxed state and the catheter mechanically captured within the first and second elongate slots such that the catheter grip may be easily slid along the axial length of the catheter.

FIG. 23 is an elevation view of the catheter grip and laser ablation catheter shown in FIG. 22 with the resilient rib of the catheter grip embodiment disposed in a relaxed state and the catheter mechanically captured within the first and second elongate slots such that the catheter grip may be easily slid along the axial length of the catheter.

FIG. 24 is a is a perspective view of the guide catheter embodiment, patient tissue, laser ablation catheter, and catheter grip embodiment shown in FIG. 13 with the catheter grip being slid along the axial length of the laser ablation catheter.

FIG. 25 is a perspective view of the guide catheter embodiment, patient tissue, laser ablation catheter, and catheter grip embodiment shown in FIG. 13 with a compression force applied to each of the respective compression regions of the catheter grip so as to elastically deform the resilient rib such that the gap between the respective distal ends of the first and second rigid plates is reduced and an outside surface of the laser ablation catheter is compressed and gripped by inside surfaces of the first and second elongate slots.

FIG. 26 is an elevation view of the guide catheter embodiment, laser ablation catheter, and catheter grip embodiment shown in FIG. 13 with a compression force applied to the respective compression regions of the catheter grip so as to elastically deform the resilient rib such that the gap between the distal ends of the first and second rigid plates is reduced and an outside surface of the catheter is compressed with inside surfaces of the first and second elongate slots.

FIG. 27 is a perspective view of the guide catheter embodiment, patient tissue, laser ablation catheter, and catheter grip embodiment shown in FIG. 13 with the catheter grip engaged with and gripping the laser ablation catheter and the catheter grip advancing the laser ablation catheter into the guide catheter embodiment.

FIG. 28 is a perspective view of a catheter grip embodiment.

FIG. 29 is an exploded perspective view of the catheter grip embodiment of FIG. 28.

FIG. 30 is a front view of the catheter grip embodiment of FIG. 28.

### DETAILED DESCRIPTION

Some catheter grip embodiments which are discussed herein may be utilized to mechanically capture, grip, and/or engage/disengage any suitable type of medical catheter which is designed to perform many and varied medical procedures such as drainage, vascular access, angioplasty including laser angioplasty, angiograms, electrophysiology procedures and the like. Some catheter grip embodiments may allow for a user of a catheter to advance, retract, and apply torque to the catheter without deforming the catheter shaft or components disposed within the shaft of the catheter.

Some catheter grip embodiments may include a rigid first plate which is pivotally secured to a rigid second plate by a resilient rib. A user of the catheter grip may apply forces to different regions of the catheter grip in order to elastically deform the rib thereby altering the shape of the catheter grip. This allows the user to mechanically capture, grip, and/or engage/disengage with the catheter in order to perform the given medical procedure without physically grasping the catheter with their hands.

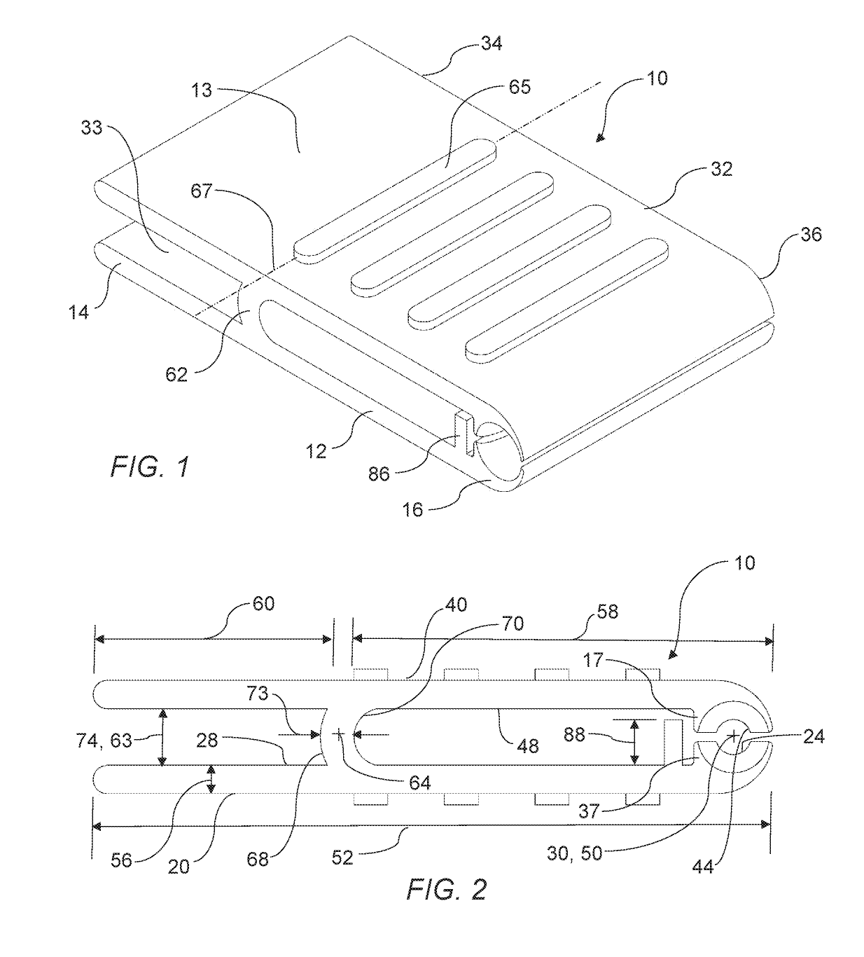

An embodiment of a catheter grip 10 is shown in FIGS. 1-3. The catheter grip 10 may include a first rigid plate 12 which has a proximal end 14, a distal end 16, and a compression region 18 on an outside surface 20 of the first rigid plate 12. The first rigid plate 12 may also have a separation region 22 which may be disposed on the outside surface 20 and be proximal to the compression region 18. The first rigid plate 12 may also include a first elongate slot 24, the inside surface of which may be formed as a cylindrical contour 26. For some embodiments, the first elongate slot 24 may extend transversely across an inside surface 28 of the distal end 16 of the first rigid plate 12. The first elongate slot 24 may also include a first longitudinal axis of curvature 30 which is located at a centroid of the transverse cylindrical contour 26.

The catheter grip embodiment 10 of FIG. 1 may also include a second rigid plate 32 which has a proximal end 34, a distal end 36, and a compression region 38 on an outside surface 40 of the second rigid plate 32. The second rigid plate 32 may also have a separation region 42 which may be disposed on the outside surface 40 and proximal to the compression region 38. The second rigid plate 32 may also include a second elongate slot 44, the surface of which may be formed as a cylindrical contour 46. For some embodiments, the second elongate slot 44 can extend transversely across an inside surface 48 of the distal end 36 of the second rigid plate 32. The second elongate slot 44 may also include a second longitudinal axis of curvature 50 which is located at a centroid of the transverse cylindrical contour 46. For some embodiments, the second elongate slot 44 may be disposed such that it is opposite to and in alignment with the first elongate slot 24.

For some embodiments of the catheter grip 10, the first and second rigid plates 12 and 32 may each have a length 52 of about 1.4 inches to about 1.6 inches. The first and second rigid plates 12 and 32 may each have a width 54 of about 0.7 inches to about 1 inch, and the first and second rigid plates 12 and 32 may each have a thickness 56 of about 0.05 inches to about 0.07 inches for some embodiments. Additionally, the length of each compression region 18 and 38 along a proximal to distal direction may be about 0.6 inches to about 0.8 inches, and the length of each separation region 22 and 42 along a proximal to distal direction may be about 0.5 inches to about 0.6 inches in some cases.

The catheter grip embodiment 10 may further include a resilient rib 62 which pivotally couples the first rigid plate 12 to the second rigid plate 32 such that respective longitudinal axes 30 and 50 of the first elongate slot 24 and second elongate slot 44 will remain substantially aligned during the pivoting at the pivot axis of the first rigid plate 12 relative the second rigid plate 32. For some catheter grip embodiments 10, the resilient rib 62 may extend from the inside surface 28 of the first rigid plate 12 to the inside surface 48 of the second rigid plate 32, thus allowing for the first rigid plate 12 and second rigid plate 32 to be displaced in a relative pivoting motion along a pivot axis 64 which is disposed such that the pivot axis 64 is parallel to the respective longitudinal axes 30 and 50 of the first and second elongate slots 24 and 44. The resilient rib 62 may also be disposed such that it is located between the respective separation region (22 and 42 respectively) and compression region (18 and 38 respectively) of each of the first and second rigid plates 12 and 32.

For some embodiments the resilient rib 62 may allow for relative pivoting motion between the first and second rigid plates 12 and 32 at the pivot axis 64, but may in turn prevent relative motion between the first and second rigid plates 12 and 32 in any other degree of freedom. For some embodiments the resilient rib 62 may have a proximal rib radius 68 of about 0.11 inches to about 0.15 inches, and may also have a distal rib radius 70 of about 0.05 inches to about 0.08 inches.

FIGS. 1-3 depict the catheter grip embodiment 10 wherein the resilient rib 62 is disposed in a relaxed state. That is to say, that no external forces are being applied to the catheter grip 10 in the relaxed state, therefore there is no externally applied elastic deformation of the resilient rib 62 in the relaxed state. When the resilient rib is disposed in the relaxed state, there are gaps between the inside surfaces 28 and 48 of the respective proximal ends 14 and 34 and distal ends 16 and 36 of the first and second rigid plates 12 and 32, and the general structure of the first and second rigid plates 12 and 32 are substantially parallel to each other for some embodiments in the relaxed state. A distal gap 72 is shown disposed between the distal ends 16 and 36 of the first and second rigid plates 12 and 32, and a proximal gap 74 is shown disposed between the proximal ends 14 and 34 of the first and second rigid plates 12 and 32 with the resilient rib 62 in a relaxed state in FIG. 2.

Additionally, the first and second longitudinal axes of curvature 30 and 50 of the respective elongate slots 24 and 44 are aligned so as to define an incomplete cylindrical bore 76. The incomplete cylindrical bore 76 may have a nominal inside diameter 82 which is slightly larger than a nominal outer diameter 84 of a catheter such as laser ablation catheter 78 which may be engaged within the incomplete cylindrical bore 76. As a specific example, for a laser ablation catheter 78 having an outer diameter 84 of 0.061 inches, the nominal inside diameter 82 of the incomplete cylindrical bore 76 may be about 0.078 inches. In this manner the first and second elongate slots 24 and 44 may be configured to mechanically capture the laser ablation catheter 78, however the incomplete cylindrical bore 76 of the catheter grip 10 is free to easily slide along the axial length of the laser ablation catheter 78 with the resilient rib 62 disposed in the relaxed state. Although the catheter grip embodiments 10 discussed herein are generally shown being used with laser ablation catheter embodiments 78 in the context of a laser ablation procedure, the catheter grip embodiments 10 may be used with any suitable catheter embodiment including but not limited to therapeutic or diagnostic catheters such as angioplasty catheters, electrophysiology ablation catheters, electrophysiology mapping catheters, drainage catheters, implant catheters, surgical catheters, atherectomy catheters, angiography catheters, urinary catheters or the like.

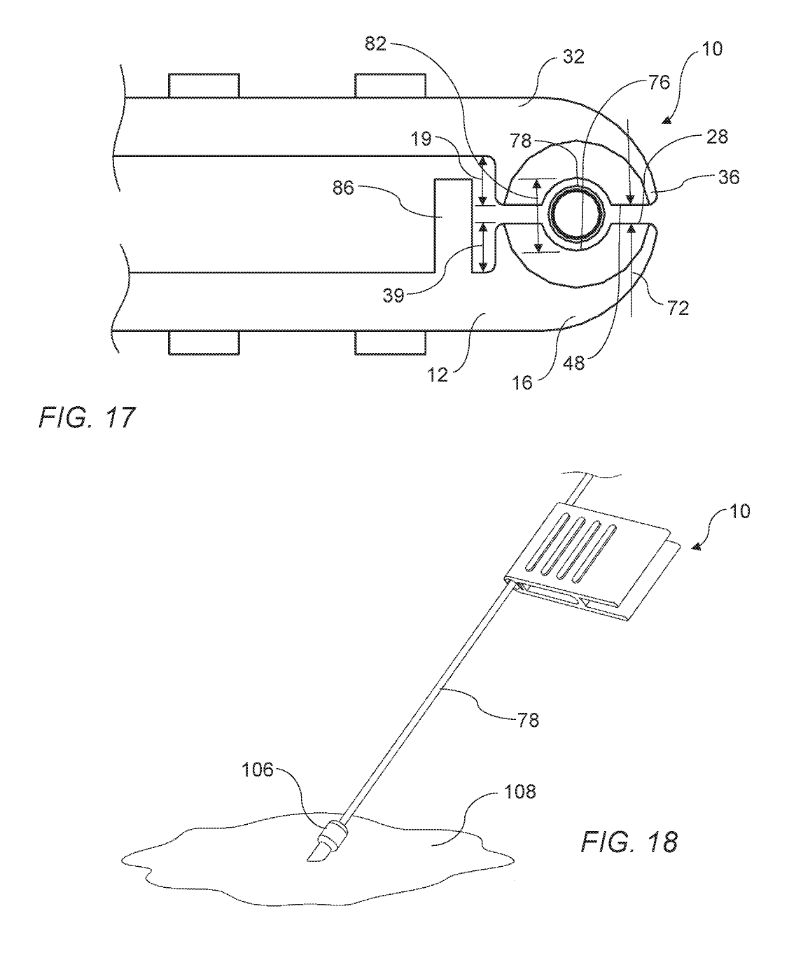

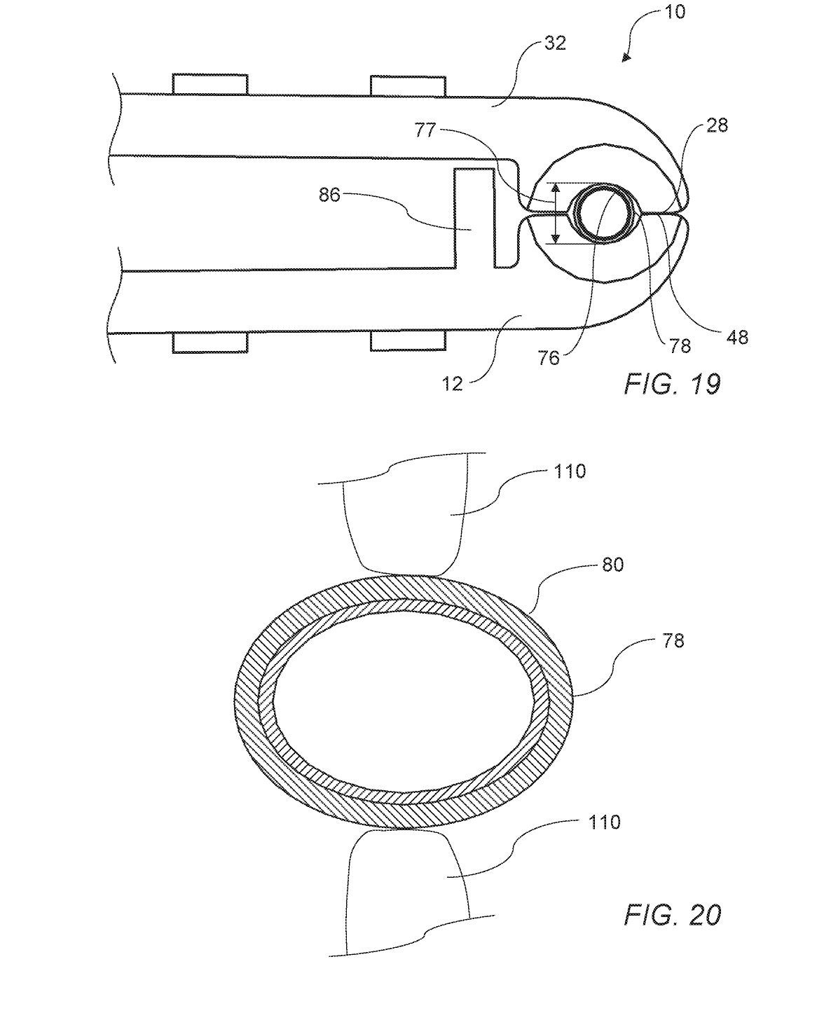

In some cases, the measurement of the gap 72 between the distal ends 16 and 36 of the first and second rigid plates 12 and 32 when resilient rib 62 is disposed in the relaxed state as shown in FIG. 17 may substantially less than the measurement of a transverse dimension 77 of the incomplete cylindrical bore 76 formed by the first and second elongate slots 24 and 44 when the resilient rib 62 is disposed in a relaxed state. The gap 72 may also be substantially less than an outside transverse dimension of the laser ablation catheter 78 with the resilient rib 62 in a relaxed state. The gap 72 when the resilient rib 62 is in a relaxed state may further be less than transverse dimension 77 when first and second compressive forces 96 and 97 have been applied to respective compressive regions 18 and 38 so as to reduce the gap 72 to zero as shown in FIG. 19.

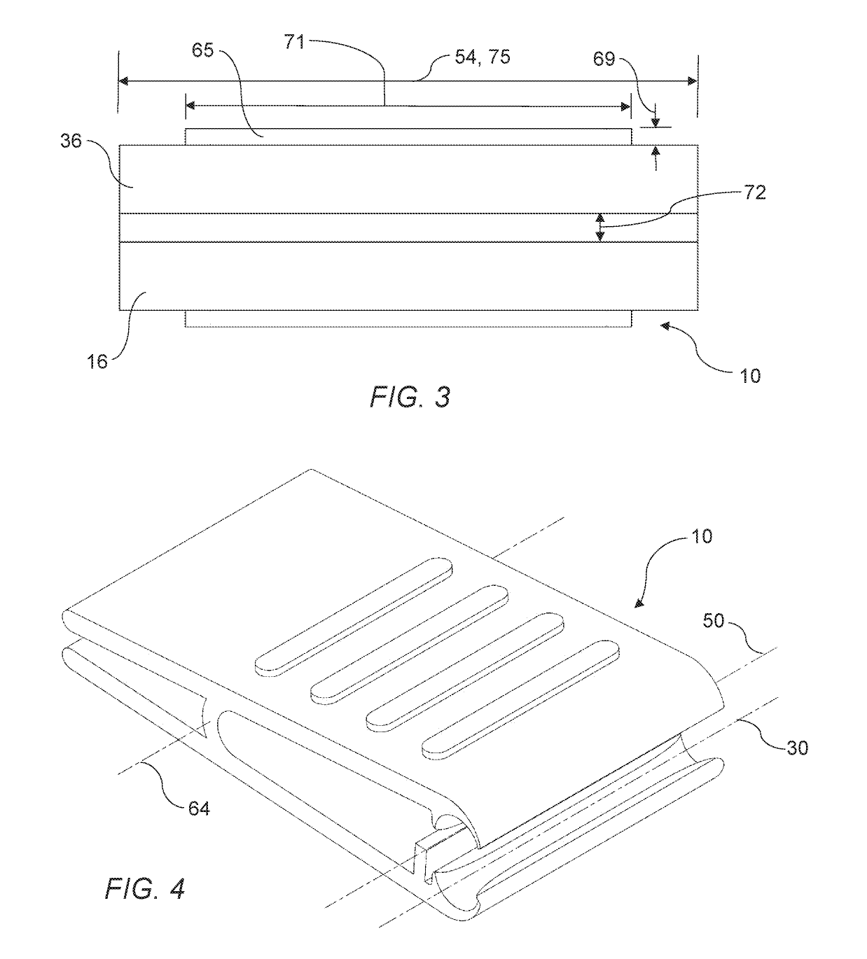

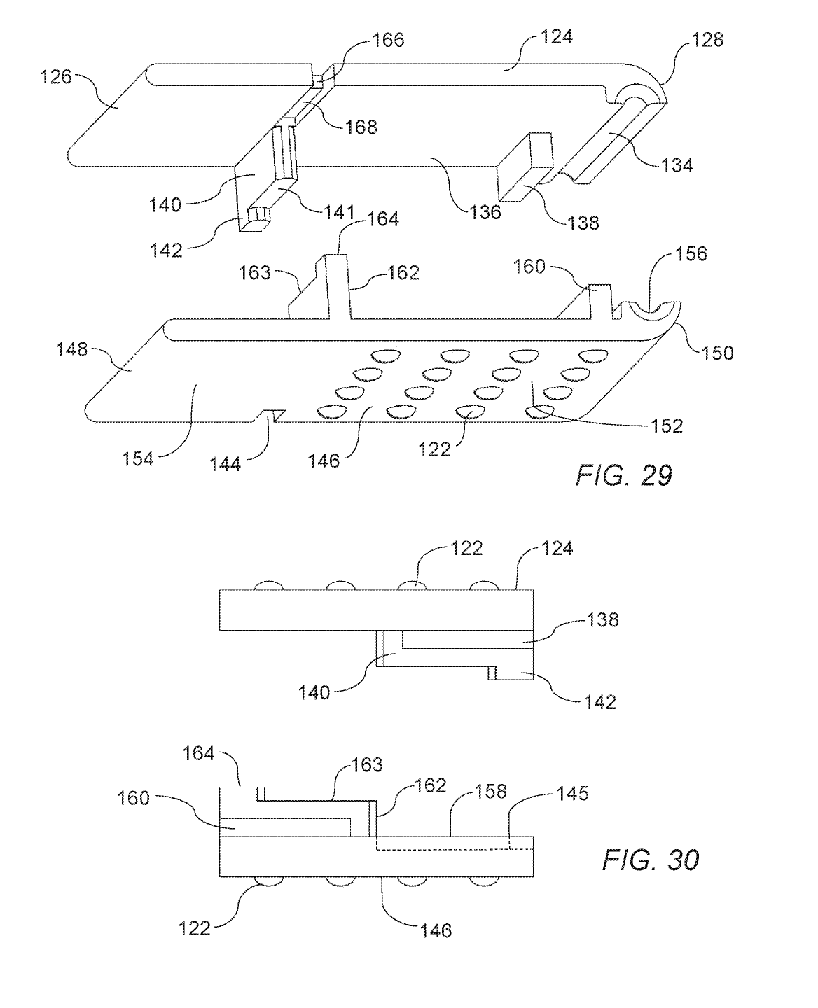

Some catheter grip embodiments 10 which are discussed herein may include a plurality of protrusions which extend from the main outside surfaces 20 and 40 of each respective compression regions 18 and 38. The protrusions may be configured to improve the grip-ability of the catheter grip 10 during manipulation of the catheter grip 10 by providing increased friction between the outside surfaces 20 and 40 of the catheter grip 10 and the fingers 110 of a user of the catheter grip 10. In some cases, each protrusion may be configured as an elongate boss 65 (see FIG. 1) which extends from a main outside surface 20 or 40 of a respective compression region 18 or 38. Each elongate boss 65 may include a longitudinal axis 67 which may be substantially parallel to the pivot axis 64. Each elongate boss 65 may have a height 69 (see FIG. 3) of about 0.01 inches to about 0.05 inches above the respective adjacent main level of the outside surface 20 or 40. Additionally, each elongate boss 65 may have a length 71 of about 0.6 inches to about 0.8 inches in some cases. Protrusion embodiments may also include semispherical protrusions (or any other suitable protrusion configuration) arranged in a rectangular grid pattern or any other suitable pattern for improved grip-ability of the catheter grip 10. The catheter grip embodiment 120 that includes such semispherical protrusions 122 is shown in FIGS. 28-30 and discussed in more detail below. For some embodiments, such semispherical protrusions may have radii of about 0.01 inches to about 0.05 inches. For some catheter grip embodiments 10, the outside surfaces 20, 40 may include about 8 semispherical protrusions to about 32 semispherical protrusions in a desired pattern.

Some embodiments of the catheter grip 10 may also include a barrier 86 that is secured to and extends from the first rigid plate 12. The barrier 86 may be disposed such that it is adjacent to the incomplete cylindrical bore 76 and parallel to the longitudinal axis of curvature 30 of the first elongate slot 24 such that it prevents the laser ablation catheter 78 from moving proximally past the elongate slots 24 and 44 during engagement of the laser ablation catheter 78 into the cylindrical bore formed by the elongate slots 24, 44. For some catheter grip embodiments 10, the height 88 of the barrier 86 may be from about 0.080 to about 0.120 inches.

For some embodiments of the catheter grip 10, the distal gap 72 between the respective distal ends 16 and 36 of the first and second rigid plates 12 and 32 with the resilient rib 62 disposed in the relaxed state may be from about 0.018 inches to about 0.022 inches, and the proximal gap 74 between the respective proximal ends 14 and 34 of the first and second rigid plates 12 and 32 with the resilient rib 62 disposed in the relaxed state may be from about 0.10 inches to about 0.15 inches. Additionally, the nominal inside diameter 82 of the incomplete cylindrical bore 76 with the resilient rib 62 disposed in the relaxed state may be from about 0.06 inches to about 0.09 inches.

FIGS. 4-6 depict the catheter grip embodiment 10 of FIG. 1 with a first separation force 94 and a second separation force 95 applied to the respective separation regions 22 and 42 of the first rigid plate 12 and the second rigid plate 32 thereby elastically deforming the resilient rib 62. Generally, the second separation force 95 and the first separation force 94 are opposed to each other in order to squeeze the separation regions 22, 42. The application of separation forces 94 and 95 serve to elastically deform the resilient rib 62 so as to expand the distal gap 72 between the respective distal ends 16 and 36 of the first and second rigid plates 12 and 32 via the resulting relative rotation of the first and second rigid plates 12 and 32 about the pivot axis 64. Such expansion of the distal gap 72 may be continued until the distal gap 72 is larger than the outside diameter or transverse dimension 84 of the laser ablation catheter 78. When the first and second separation forces 94 and 95 are applied to the respective separation regions 22 and 42, the laser ablation catheter 78 may be engaged to or disengaged from the elongate slots 24 and 44 without any deformation of the laser ablation catheter 78. For some embodiments the first and second separation forces 94 and 95 applied to the respective compression regions 18 and 38 required to elastically deform the resilient rib 62 and expand the distal gap 72 between the distal ends 16 and 36 of the first and second rigid plates 12 and 32 may be from about 13 Newtons to about 17 Newtons each.

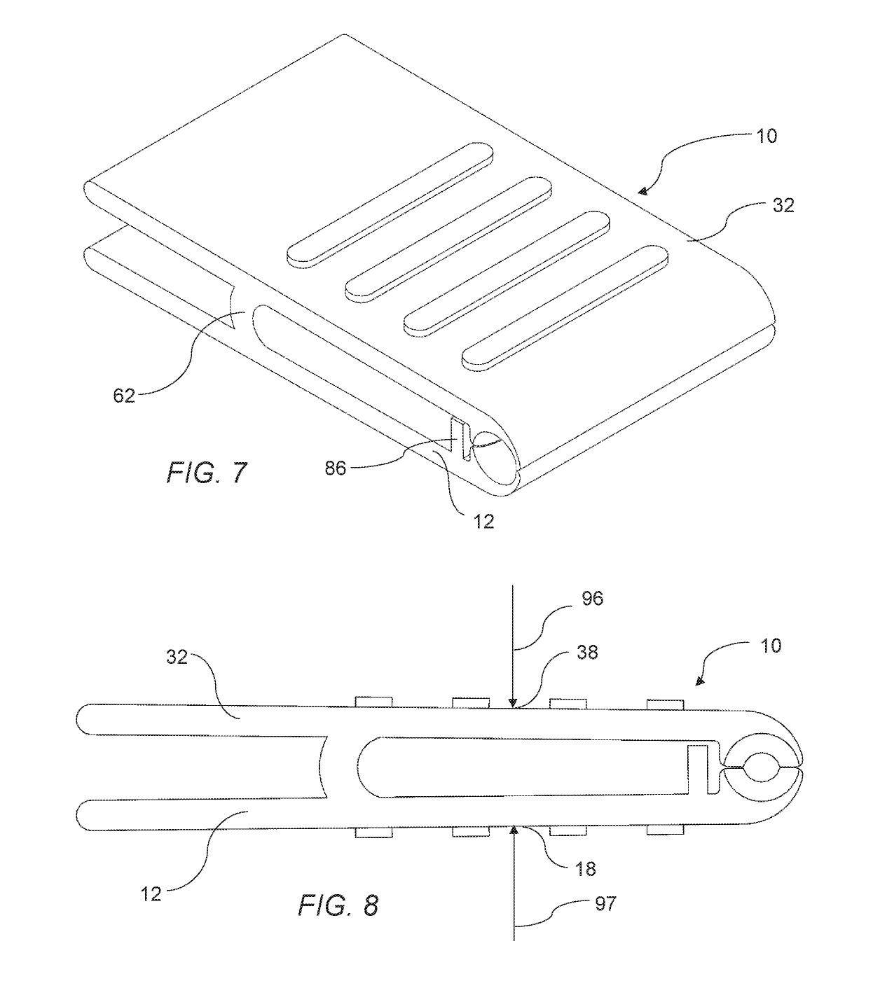

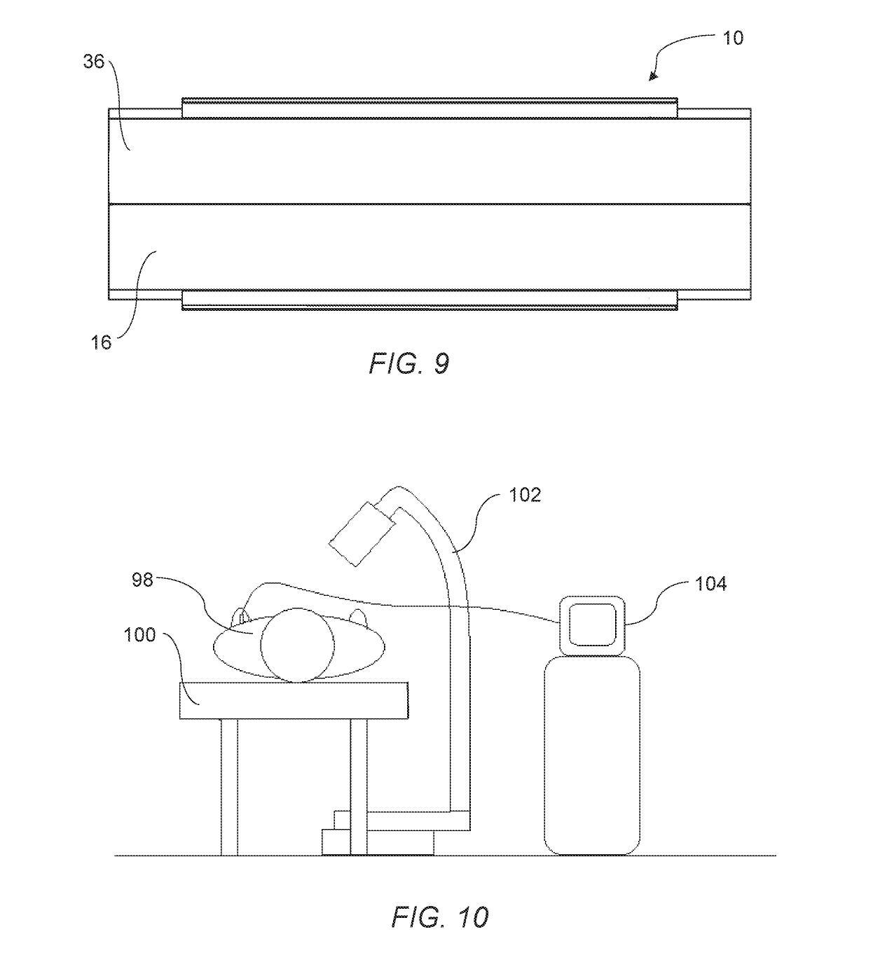

FIGS. 7-9 depict the catheter grip embodiment 10 of FIG. 1 with a first compressive force 96 and a second compressive force 97 applied to the respective compression regions 18 and 38 of the first rigid plate and the second rigid plate 12 and 32 thereby elastically deforming the resilient rib 62. As discussed above, the second compression force 97 and first compression force 96 are generally opposed to each other as shown in FIG. 8. The first and second compressive forces 96 and 97 reduce the distal gap 72 between the respective distal ends 16 and 36 of the first and second rigid plates 12 and 32 via rotation of the first and second rigid plates 12 and 32 about the pivot axis 64. The reduction in the distal gap 72 allows for the gripping of the laser ablation catheter 78 via the compression of the outside surface 80 of the laser ablation catheter 78 with the respective inside surfaces 26 and 46 of the first and second elongate slots 24 and 44. For some embodiments the first and second compressive forces 96 and 97 applied to the respective separation regions 22 and 42 required to elastically deform the resilient rib 62 and reduce the distal gap 72 between the distal ends 16 and 36 of the first and second rigid plates 12 and 32 may be from about 13 Newtons to about 17 Newtons each.

The catheter grip 10 may be fabricated by any suitable means. For some embodiments, the first rigid plate 12, second rigid plate 32, and resilient rib 62 include a monolithic structure formed from a single piece of continuous uninterrupted material. The monolithic structure may be formed by processes such as injection molding, 3-D printing, machining, or the like. The catheter grip embodiments 10 discussed herein may also be formed from multiple components made from any of the processes discussed above. An example of such a catheter grip embodiment 120 is shown in FIGS. 28-30 and discussed below. The multiple components of such embodiments may be secured together by any suitable means such as solvent welding, ultrasonic welding, thermal welding, adhesive bonding or the like. For solvent welding, solvents such as acetone or any other suitable solvent that dissolves the material of the catheter grip 10 may be used. In some cases, the catheter grip 10 and components thereof including the first rigid plate 12, second rigid plate 32, and resilient rib 62 may include a high strength resilient polymer such as medical grade ABS (acrylonitrile-butadiene-styrene) plastic, polyvinylchloride (PVC), nylon, polycarbonate or the like.

As discussed above the catheter grip 10 may include the first rigid plate 12 which is pivotally secured to the second rigid plate 32 by the resilient rib 62, thereby allowing for pivotal motion of the first and second rigid plates 12 and 32 about a pivot axis 64. The first rigid plate 12 may include a first plate body 13, the first plate body 13 having a substantially rectangular shape which may be defined by the plate length 52 and plate width 54 as shown in FIGS. 2 and 3. For some embodiments the plate length 52 may be longer than the plate width 54, and the plate thickness 56 (see FIG. 2) may be much smaller than the plate width 54 in order to minimize the overall profile of the catheter grip 10. For some first rigid plate embodiments 12 which are discussed herein, in addition to the rectangular shape discussed above, the first plate body 13 may be configured to have any suitable shape such as an oval shape, elliptical shape or the like.

The second rigid plate 22 may include a second plate body 33, the second plate body 33 having a substantially rectangular shape which may be defined by the plate length 52 and plate width 54. For some embodiments the plate length 52 may be longer than the plate width 54, and the plate thickness 56 may be much smaller than the plate width 54 in order to minimize the overall profile of the catheter grip 10. For some second rigid plate embodiments 32 which are discussed herein, in addition to the rectangular shape discussed above, the second plate body may have as any suitable shape such as an oval shape, elliptical shape or the like. In some cases the shape and dimensions of the second plate body 33 may be configured to substantially similar to the shape and dimensions (e.g. plate length 52, plate width 54, and plate thickness 56 for rectangular plate body shapes) of the first plate body 13. That is to say that a first plate body 13 having a rectangular shape may be operatively coupled to a second plate body 33 having a similar rectangular shape and similar dimensions by the resilient rib 62. Similarly, a first plate body 13 having an elliptical shape may be operatively coupled to a second plate body 33 having a similar elliptical shape and similar dimensions by the resilient rib 62.

Both first rigid plate 12 and the second rigid plate 32 may include multiple surfaces with each surface being configured to optimize user interaction and/or functionality of the catheter grip 10. In some cases the outside surfaces 20 and 40 and their associated features may be configured to optimize the user interaction of the catheter grip 10, while the inside surfaces 28 and 48 and their associated features may be configured to optimize the functionality of the catheter grip 10. The outside surface 20 of the first rigid plate 12 may include a surface of the planar compression region 18 and a surface of the planar separation region 22, with the separation region 22 disposed proximal to the compression region 18 and the resilient rib 62 disposed between the compression and separation regions 18 and 22. For some first rigid plate embodiments 12, the compression region 18 may have a length 58 (see FIG. 2) which is longer than a length 60 of the separation region 22. For such embodiments the increased length 58 of the compression region 18 allows for an increased bending moment about the pivot axis 64 of the resilient rib 62 during the application of compressive forces 96 to the compression region 18 thereby providing a mechanical advantage during gripping of the laser ablation catheter 78. In some cases, the separation region 42 may have a shorter length 50 than the length 58 of the compression region 38. In some instances, this may be because less mechanical advantage may be required to insert/release the laser ablation catheter 78 into the partial cylindrical bore of the catheter grip 10 under the application of separation forces 94 and 95 than mechanical advantage required to grip the laser ablation catheter 78 with the catheter grip 10. The lengths of the compression and separation regions 38 and 42 of the second rigid plate 32 may be similarly configured in order to provide a desired mechanical advantage during the application of the compressive forces 96 and 97 as discussed above.

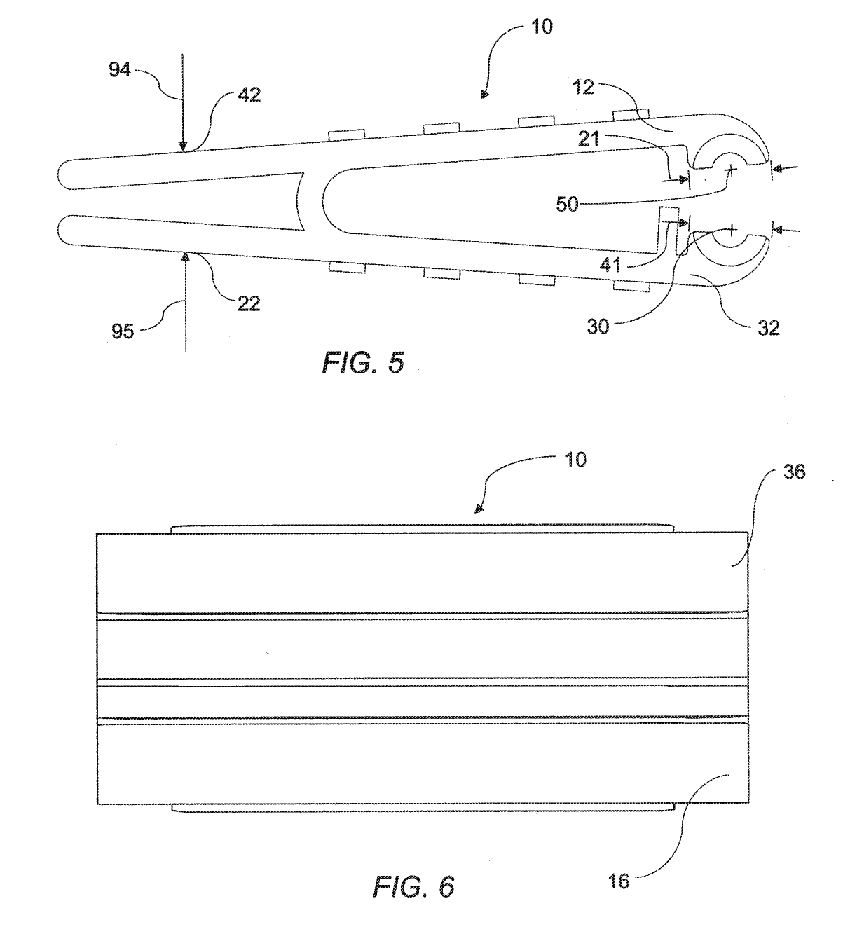

Some embodiments of the first rigid plate 12 may have an optional first distal boss 17 as shown in FIG. 2 which may extend from the inside surface 28 of the distal end 16 of the first rigid plate 12. The first distal boss 17 may also extend transversely across the distal end 16 such that it spans the plate width 54 of the first rigid plate 12. In some cases the first distal boss 17 may have a height 19 which extends about 0.04 inches to about 0.06 inches inward from the inside surface 28 of the first rigid plate 12. The first distal boss 17 may also have a thickness 21 (see FIG. 5) extending along a proximal to distal direction of about 0.15 inches to about 0.19 inches. Similarly some embodiments of the second rigid plate 32 may have an optional second distal boss 37 which may extend from the inside surface 48 of the distal end 36 of the second rigid plate 32. The second distal boss 37 may also extend transversely across the distal end 36 such that it spans the plate width 54 of the second rigid plate 32. In some cases the second distal boss 37 may have a height 39 which extends of about 0.04 inches to about 0.06 inches inward from the inside surface 48 of the second rigid plate 32. The second distal boss 37 may also have a thickness 41 extending along a proximal to distal direction of about 0.15 inches to about 0.19 inches.

The resilient rib 62 which extends from the inside surface 28 of the first rigid plate 12 to the inside surface 48 of the second rigid plate 32 may be configured to allow for resilient displacement of the first rigid plate 12 and second rigid plate 32 in a relative pivoting motion about the pivot axis 64. For such resilient displacement, any relative rotational displacement between the first rigid plate 12 and the second rigid plate 32 from the relaxed state of the resilient rib 62 is elastically resisted by the elasticity of the material of the resilient rib 62 such that when external compressive forces are released, the first and second rigid plates 12 and 32 will return to the original position of the relaxed state. The application of the compression forces 96 and 97 or of the separation forces 94 and 95 to the respective compression regions 18 and 38 or separation regions 22 and 42 of the catheter grip 10 results in a bending moment about the pivot axis 64 of the resilient rib 62 which in turn leads to the elastic deformation of the resilient rib 62. The shape of the resilient rib 62 may therefore allow for the pivotal motion of the first and second rigid plates 12 and 32 with minimized stresses applied to the resilient rib 62 under the application of bending moments. The resilient rib 62 may have a rib height 63, a rib thickness 73, (see FIG. 2) and a rib width 75 with the pivot axis 64 extending along the rib width 75 (see FIG. 3). In some cases the rib thickness 73 may be much smaller than the rib width 75 in order to allow for elastic deformation of the resilient rib 62 upon application of the compression forces 96 and 97 or of the separation forces 94 and 95 to the respective compression regions 18 and 38 or separation regions 22 and 42 of the catheter grip 10. Additionally some surfaces of resilient rib 62 which are substantially parallel to the pivot axis 64 may be configured with radii in order to reduce the internal stresses applied to the resilient rib 62 under the application of bending moments. FIG. 2 depicts such surfaces of the resilient rib 62, the proximal rib radius 68 and the distal rib radius 70.

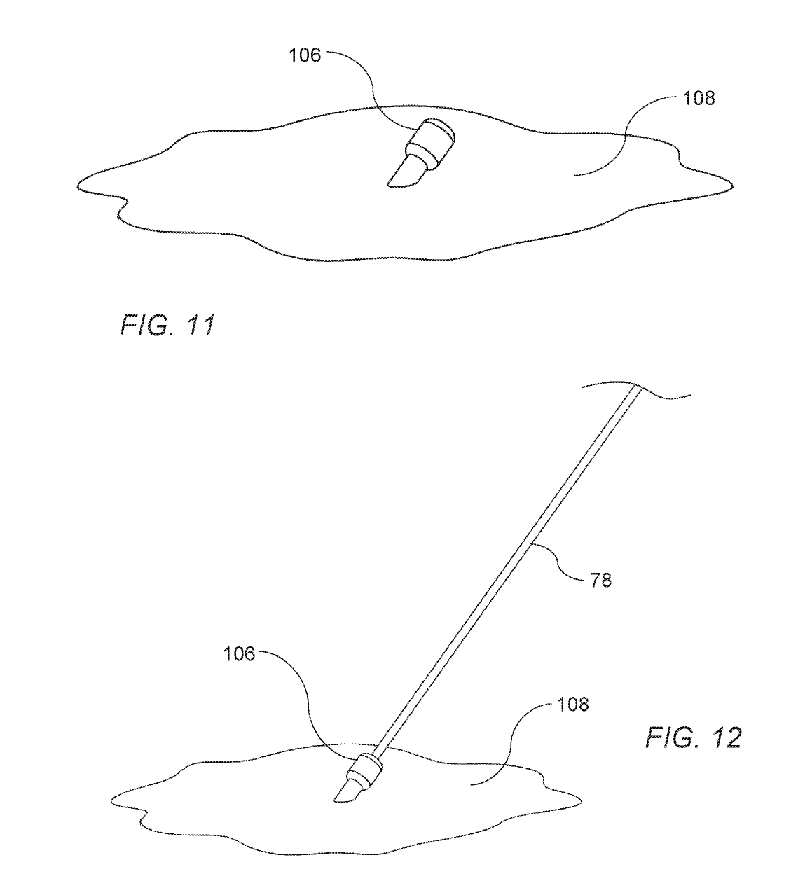

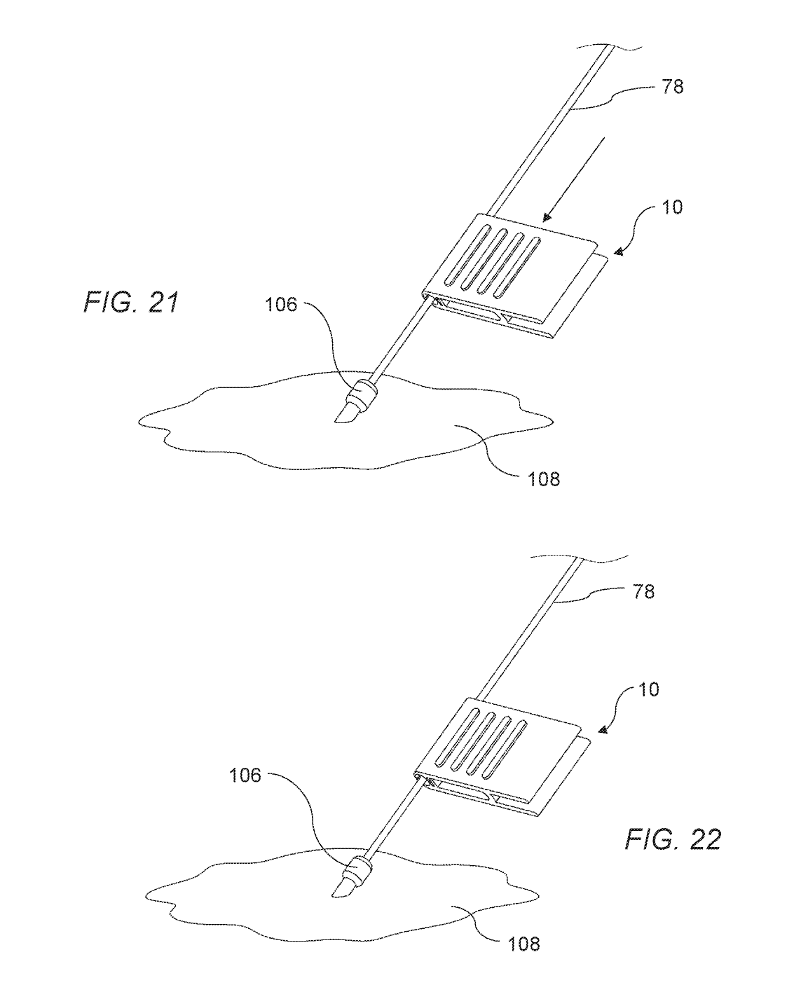

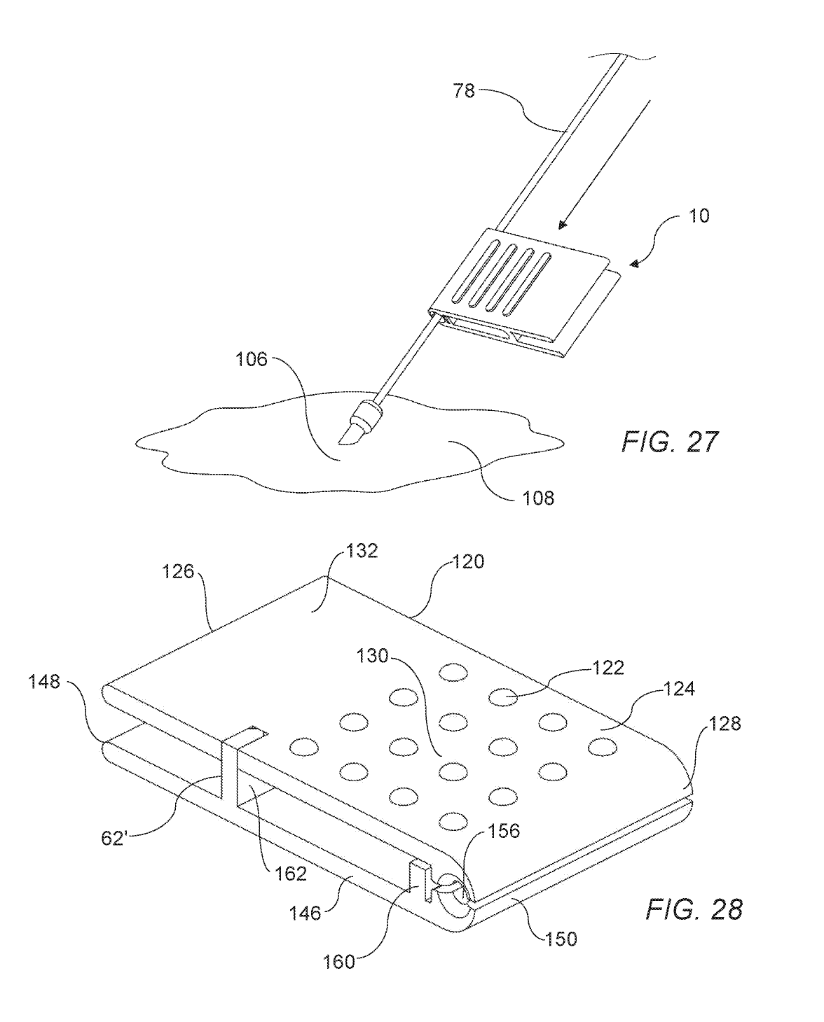

FIGS. 10-27 depict the use of an embodiment of a catheter grip 10 during a medical laser ablation catheter procedure. FIG. 10 depicts a patient 98 on a table 100, an imaging fluoroscope 102, a medical laser 104, and a medical catheter, which for the embodiment shown is a laser ablation catheter 78. The procedure is initiated with the insertion of a guide catheter 106 into a body lumen of the patient 98. In some cases, the guide catheter embodiments 106 which are discussed herein may include any catheter configuration or guiding accessory suitable for insertion of laser ablation catheter 78 into a patient's body lumen. Such catheter configurations and guiding accessories may include introducers, sheaths, standard guiding catheters or the like. FIG. 11 depicts the inserted guide catheter 106 and the surface of the tissue 108 of the patient 98. The laser ablation catheter 78 is then inserted into the guide catheter 106 as shown in FIG. 12. The guide catheter 106 is typically designed to be hemostatic and therefore may incorporate a flexible gland which surrounds the laser ablation catheter 78.

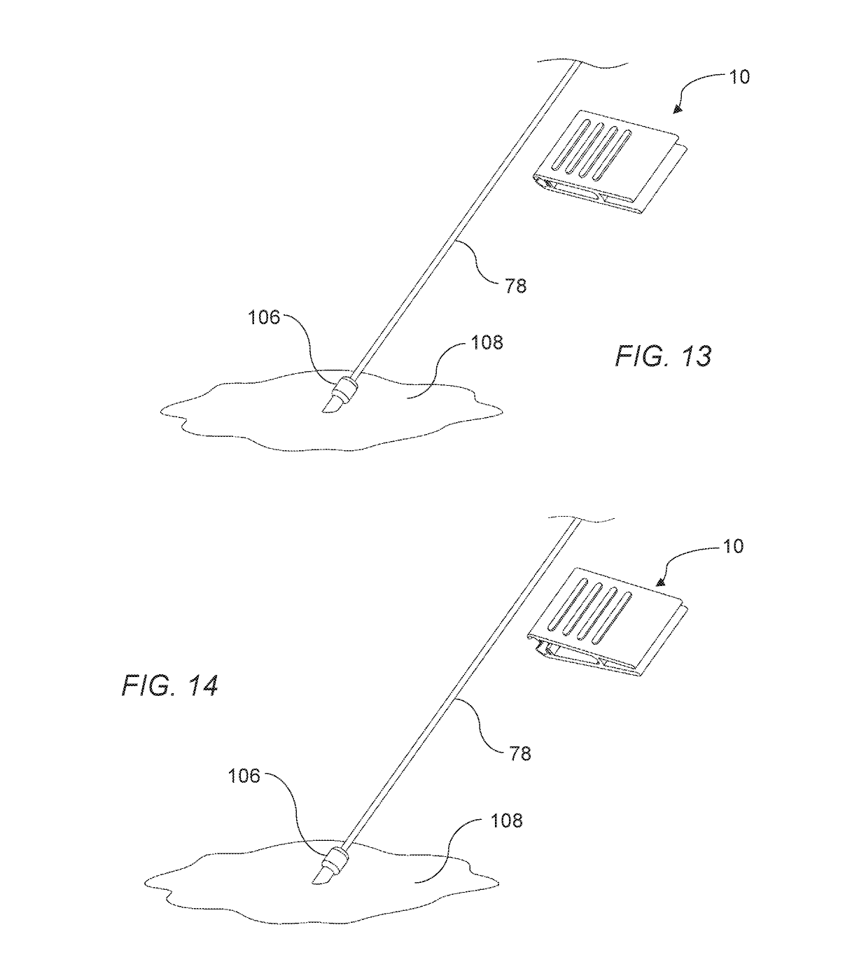

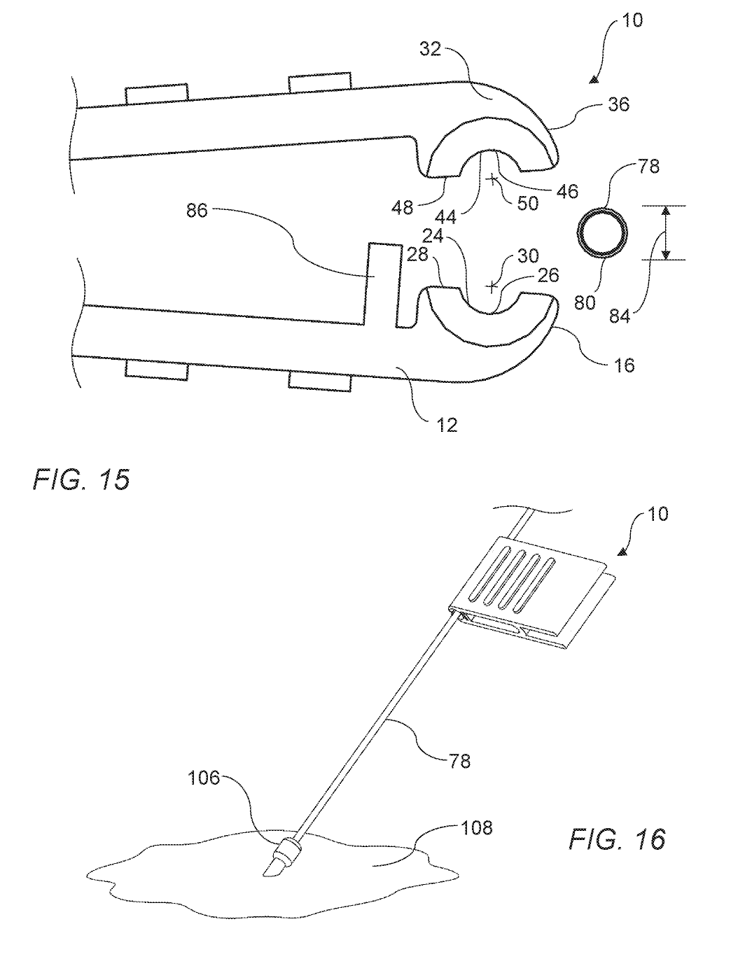

After insertion of the laser ablation catheter 78 into the guide catheter 106, the catheter grip 10 may be moved into position next to the laser ablation catheter 78 as shown in FIG. 13. First and second separation forces 94 and 95 may then be applied to the respective separation regions 22 and 42 of the first and second rigid plates 12 and 32 (see FIG. 5) until the distal gap 72 between the distal ends 16 and 36 of the first and second rigid plates 12 and 32 is greater than the outside diameter 84 of the laser ablation catheter 78 as shown in FIG. 15. This allows for the engagement of the catheter grip 10 with the laser ablation catheter 78 without deformation of the laser ablation catheter 78 by inserting the laser ablation catheter 78 through the distal ends 16 and 36 of the first and second plates 12 and 32.

At this point, the first and second separation forces 94 and 95 may be released thereby transitioning the resilient rib 62 to the relaxed state wherein the first and second rigid plates 12 and 32 are substantially parallel to each other. Additionally, the first and second longitudinal axes of curvature 30 and 50 of the respective elongate slots 24 and 44 may be aligned so as to define the incomplete cylindrical bore 76 with a nominal inside diameter 82 slightly larger than a nominal outer diameter 84 of the laser ablation catheter 78 as shown in FIGS. 16-17.

The catheter grip 10 may then be utilized to grip the laser ablation catheter 78 by applying first and second compressive forces 96 and 97 to the respective compression regions 18 and 38 of the first and second rigid plates 12 and 32 (see FIG. 8). The first and second compressive forces 96 and 97 elastically deform the resilient rib 62 and reduce the distal gap 72 between the distal ends 16 and 36 of the first and second rigid plates 12 and 32 thereby compressing the outside surface 80 of the laser ablation catheter 78 with the respective inside surfaces 26 and 46 of the first and second elongate slots 24 and 44 as shown in FIGS. 18 and 19.

The catheter grip 10 provides a superior connection to the laser ablation catheter 78 when compared to gripping the laser catheter 78 with fingers 110, which is depicted in FIG. 20. Gripping the laser ablation catheter 78 with fingers 110 may lead to distortion of the body of the laser ablation catheter 78 as shown in FIG. 20. This may be due to the gripping force being applied to the laser ablation catheter 78 by fingers 110 unevenly or too forcefully at multiple contact points of the outside surface 80 of the laser ablation catheter 78. This is in contrast to the catheter grip embodiment 10 depicted in FIG. 19 wherein the laser ablation catheter 78 is engaged evenly over a larger surface area of the outer surface 80 of the laser ablation catheter 78. As discussed above, distortion or deformation of a medical catheter, and particularly, a medical laser ablation catheter 78, may reduce the performance of the laser ablation catheter 78. In addition, in some cases, laser ablation catheter embodiments 78 may include a liquid core waveguide configuration that may be particularly susceptible to reduced performance as a result of deformation caused by uneven or overly forceful gripping of the outer surface of the laser ablation catheter 78. In some instances, laser energy transmission by the laser ablation catheter 78 may be negatively affected by such deformation. Embodiments of such laser ablation catheters including liquid core waveguide configurations are discussed in U.S. Pat. No. 9,700,655, filed Oct. 12, 2012, by J. Laudenslager et al., titled “Small Flexible Liquid Core Catheter for Laser Ablation in Body Lumens and Methods for Use”, which is incorporated by reference herein in its entirety.

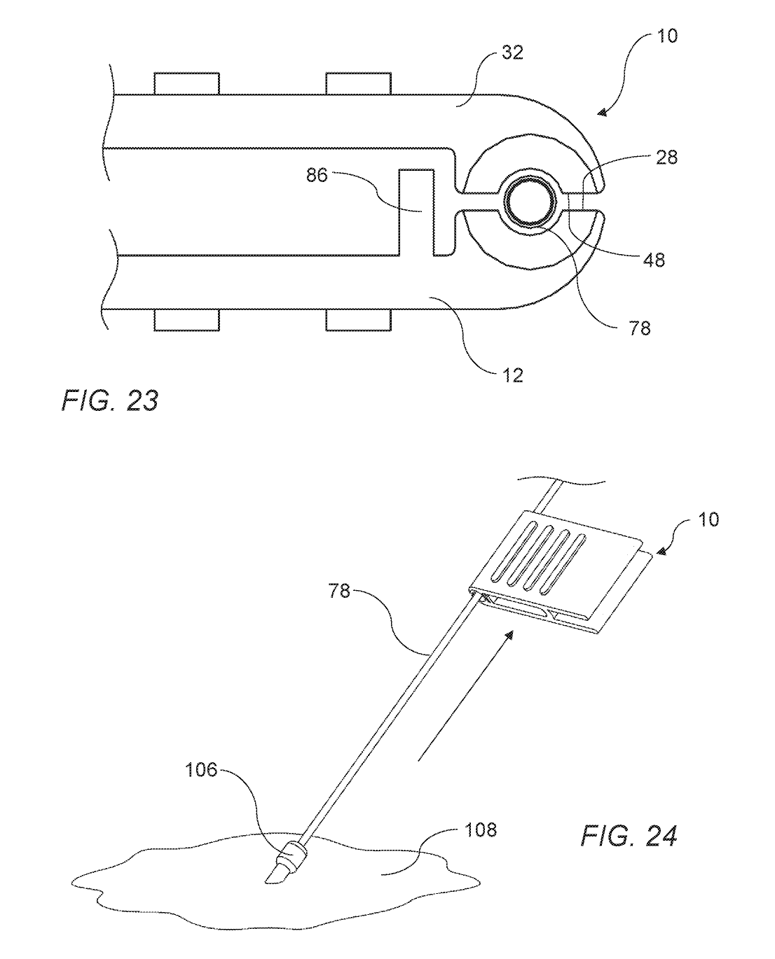

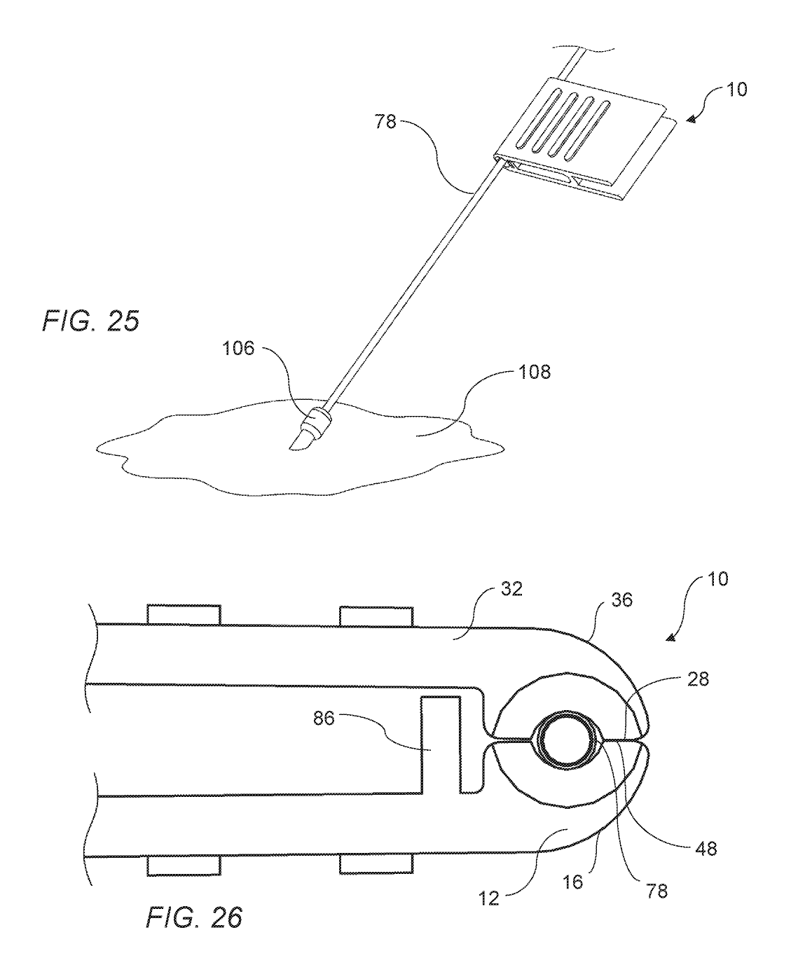

The laser ablation catheter 78 may then be translated into the patient 98 utilizing the catheter grip 10 as shown in FIG. 22. The first and second compressive forces 96 and 97 may then be removed thereby transitioning the resilient rib 62 to the relaxed state as shown in FIGS. 22 and 23. At this point the catheter grip 10 is engaged in the incomplete cylindrical bore 76 such that the first and second elongate slots 24 and 44 mechanically capture the laser ablation catheter 78, but the catheter grip 10 may be easily slid along the axial length of the laser ablation catheter 78 by applying an axial force onto the laser ablation catheter as shown in FIG. 24 wherein the catheter grip 10 has been positioned to advance more of the laser ablation catheter 78 into the patient 98. FIGS. 25-27 depict the advancement of the laser ablation catheter 78 into the patient 98 as has been discussed previously, the process being repeated until the laser ablation catheter 78 is advanced to its target within the patient 98. This catheter advancement procedure using the catheter grip 10 may also be reversed in order to remove the laser ablation catheter 78 from the patient 98.

As discussed above, the catheter grip embodiments discussed and contemplated herein may be made from a variety of suitable materials and manufactured by a variety of suitable methods. In some cases, the catheter grip embodiment 10 may be made from a single piece of continuous uninterrupted material with a monolithic configuration as discussed above. FIGS. 28-30 illustrate a catheter grip 120 that is manufactured from two separate subcomponents although the catheter grip 120 may otherwise include dimensions, materials and features which are the same or similar to the dimensions, materials and features of the catheter grip embodiment 10.

The catheter grip 120 includes a first rigid plate 124 having a proximal end 126, a distal end 128, a compression region 130 on an outside surface of the first rigid plate 124 and a separation region 132 on the outside surface proximal of the compression region 130. The first rigid plate 124 also has a first elongate slot 134 with a cylindrical contour extending transversely across an inside surface 136 at the distal end 128, the first elongate slot further including a first longitudinal axis 30 of curvature. A first barrier 138 extends inwardly from the inside surface 136 proximal of and adjacent to the first elongate slot 134 and extends transversely across a partial width of the first rigid plate 124. A first rib 140 extends inwardly from the inside surface 136 between the compression region 130 and separation region 132 and extends transversely across a partial width of the first rigid plate 124. The first rib 140 further includes a first rib extension 142 that is sized and positioned to be inserted into a corresponding first rib extension slot 144 in a second rigid plate 146. The first rib extension 142 and first rib extension slot 144 may be sized for a close fit between an inside surface of the first rib extension slot 144 and an outside surface of the first rib extension 142. In addition, an inward end 141 of the first rib 140 and first rib slot 145 of the second rigid plate 146 may also be sized for a close fit between an inside surface of the first rib slot 145 and an outside surface the inward end 141 of the first rib 140.

The second rigid plate 146 has a proximal end 148, a distal end 150, a compression region 152 on an outside surface of the first rigid plate 146 and a separation region 154 on the outside surface proximal of the compression region 152. The second rigid plate 146 also has a second elongate slot 156 with a cylindrical contour extending transversely across an inside surface 158 at the distal end 150, the second elongate slot further including a second longitudinal axis 50 of curvature. A second barrier 160 extends inwardly from the inside surface 158 proximal of and adjacent to the second elongate slot 156 and extends transversely across a partial width of the second rigid plate 146. A second rib 162 extends inwardly from the inside surface 158 between the compression region 152 and separation region 154 and extends transversely across a partial width of the second rigid plate 146. The second rib 162 further includes a second rib extension 164 that is sized and positioned to be inserted into a corresponding second rib extension slot 166 in the second rigid plate 146. The second rib extension 164 and second rib extension slot 166 may be sized for a close fit between an inside surface of the first rib extension slot 166 and an outside surface of the first rib extension 164. In addition, an inward end 163 of the second rib 162 and second rib slot 168 of the first rigid plate 124 may also be sized for a close fit between an inside surface of the second rib slot 168 and an outside surface of the inward end 163 of the second rib 162. During assembly of the catheter grip embodiment 120, the outside surfaces of the inward ends 141, 163 of the first rib 140 and second rib 162 may be secured to corresponding first and second rib slots 145, 168 by any suitable means such as solvent welding, ultrasonic welding, thermal welding, adhesive bonding or the like. For solvent welding, solvents such as acetone or any other suitable solvent that dissolves the material of the first rigid plate 124 and second rigid plate 146 of the catheter grip 120 may be used. The outside surfaces of the rib extensions 142, 164 may also be so secured to corresponding inside surfaces of the respective first and second rib extension slots 144, 166.

Once the first rigid plate 124 is so secured to the second rigid plate 146, the first rib 140 and second rib 162 form a rib 62′ that extends transversely across a transverse width or substantial portion thereof of the catheter grip 120. The rib 62′ then serves to pivotally couple the first rigid plate 124 to the second rigid plate 146 such that respective longitudinal axes 30 and 50 of the first elongate slot 134 and second elongate slot 156 remain aligned during pivoting of the first rigid plate 124 relative to the second rigid plate 146. Also, once the catheter grip 120 is assembled, the rib 62′ extends from the inside surface 136 of the first rigid plate 124 to the inside surface 158 of the second rigid plate 146 which allows the first rigid plate 124 and second rigid plate 146 to be displaced in a relative pivoting motion along a pivot axis 64 which is parallel to the respective longitudinal axes 30 and 50 of the first and second elongate slots 134, 156. The pivot axis 64 is also disposed between the respective separation regions 132, 154 and compression regions 130, 152 of each of the first and second rigid plates 124, 146.

Embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation and use of such terms and expressions do not exclude any equivalents of the features shown and described or portions thereof, and various modifications are possible. The term “a” or “an” can refer to one of or a plurality of the elements it modifies (e.g., “a reagent” can mean one or more reagents) unless it is contextually clear either one of the elements or more than one of the elements is described. Thus, it should be understood that although embodiments have been specifically disclosed by representative embodiments and optional features, modification and variation of the concepts herein disclosed may be resorted to by those skilled in the art, and such modifications and variations are considered within the scope of this disclosure.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

Certain embodiments of the technology are set forth in the claim(s) that follow(s).

What is claimed is:

1. A catheter grip, comprising:
a first rigid plate including a proximal end, a distal end, a compression region on an outside surface of the first rigid plate, a separation region on the outside surface proximal of the compression region and a first elongate slot with a cylindrical contour extending transversely across an inside surface of the distal end, the first elongate slot including a first longitudinal axis of curvature;
a second rigid plate including a proximal end, a distal end, a compression region on an outside surface of the second rigid plate, a separation region on the outside surface of the second rigid plate proximal of the compression region and a second elongate slot with a cylindrical contour extending transversely across an inside surface of the distal end of the second rigid plate, the second elongate slot including a second longitudinal axis of curvature, the second elongate slot being disposed opposite to and in alignment with the first elongate slot; and
a resilient rib which pivotally couples the first rigid plate to the second rigid plate such that respective longitudinal axes of the first elongate slot and second elongate slot remain aligned during pivoting of the first rigid plate and second rigid plate, which extends from the inside surface of the first rigid plate to the inside surface of the second rigid plate; which allows the first rigid plate and second rigid plate to be displaced in a relative pivoting motion along a pivot axis which is parallel to the respective longitudinal axes of the first and second elongate slots, and which is disposed between the respective separation region and compression region of each of the first and second rigid plates.

2. The catheter grip of claim 1 wherein a gap is disposed between the inside surfaces of the proximal ends and a gap is disposed between the inside surfaces of the distal ends of the first and second rigid plates when the resilient rib is in a relaxed state.

3. The catheter grip of claim 2 wherein the first and second rigid plates are substantially parallel to each other when the resilient rib is in a relaxed state.

4. The catheter grip of claim 3 wherein the first and second longitudinal axes of curvature of the respective elongate slots are aligned so as to define an incomplete cylindrical bore with a nominal inside diameter slightly larger than a nominal outer diameter of a catheter engaged in the incomplete cylindrical bore such that the first and second elongate slots mechanically capture a catheter but may be easily slid along an axial length of the catheter when the resilient rib is in a relaxed state.

5. The catheter grip of claim 4 wherein the catheter when engaged may be gripped by the application of a compressive force to the respective compression regions of the first rigid plate and second rigid plate so as to elastically deform the rib, reduce the gap disposed between the distal ends of the first and second rigid plates and compress the outside surface of the catheter with respective inside surfaces of the first and second elongate slots.

6. The catheter grip of claim 5 wherein the catheter may be engaged or disengaged from the elongate slots without deformation of the catheter by the application of a separation force to the respective separation regions so as to expand the gap between the distal ends until the gap is greater than the outside diameter of the catheter.

7. The catheter grip of claim 1 further comprising a plurality of protrusions extending from a main outside surface of each compression region, the protrusions being configured to improve the grip-ability of the catheter grip during manipulation.

8. The catheter grip of claim 7 wherein each protrusion comprises an elongate boss extending from the main outside surface of the respective compression region, a longitudinal axis of each boss being substantially parallel to the pivot axis.

9. The catheter grip of claim 8 wherein each elongate boss has a height of about 0.01 to about 0.05 inches above an adjacent main outside surface.

10. The catheter grip of claim 8 wherein each elongate boss has a length of about 0.6 to about 0.8 inches.

11. The catheter grip of claim 1 wherein the first rigid plate and the second rigid plate each have a length of about 1.4 to about 1.6 inches.

12. The catheter grip of claim 1 wherein the first rigid plate and the second rigid plate each have a width of about 0.7 inches to about 1.0 inches.

13. The catheter grip of claim 1 wherein the first rigid plate and the second rigid plate each have a thickness of about 0.05 to about 0.07 inches.

14. The catheter grip of claim 1 wherein each compression region has a length along a proximal to distal direction of about 0.6 to about 0.8 inches.

15. The catheter grip of claim 1 wherein each separation region has a length along a proximal to distal direction of about 0.5 to about 0.6 inches.

16. The catheter grip of claim 1 wherein the catheter grip comprises a monolithic structure formed from a continuous uninterrupted material.

17. The catheter grip of claim 16 wherein the material is a resilient polymer.

18. The catheter grip of claim 17 wherein the resilient polymer is acrylonitrile-butadiene-styrene.

19. A catheter grip, comprising:
a first rigid plate including a proximal end, a distal end, a compression region on an outside surface of the first rigid plate, a separation region on the outside surface proximal of the compression region and a first elongate slot with a cylindrical contour extending transversely across an inside surface of the distal end, the first elongate slot including a first longitudinal axis of curvature;
a second rigid plate including a proximal end, a distal end, a compression region on an outside surface of the second rigid plate, a separation region on the outside surface of the second rigid plate proximal of the compression region and a second elongate slot with a cylindrical contour extending transversely across an inside surface of the distal end of the second rigid plate, the second elongate slot including a second longitudinal axis of curvature, the second elongate slot being disposed opposite to and in alignment with the first elongate slot;
a resilient rib which pivotally couples the first rigid plate to the second rigid plate such that respective longitudinal axes of the first elongate slot and second elongate slot remain aligned during pivoting of the first rigid plate and second rigid plate, which extends from the inside surface of the first rigid plate to the inside surface of the second rigid plate; which allows the first rigid plate and second rigid plate to be displaced in a relative pivoting motion along a pivot axis which is parallel to the respective longitudinal axes of the first and second elongate slots, and which is disposed between the respective separation region and compression region of each of the first and second rigid plates;
wherein when the resilient rib is in a relaxed state, there is a gap between the inside surfaces of the proximal ends and a gap between the inside surfaces of the distal ends of the first and second rigid plates, the first and second rigid plates are substantially parallel to each other, the first and second longitudinal axes of curvature of the respective elongate slots are aligned so as to define an incomplete cylindrical bore with a nominal inside diameter slightly larger than a nominal outer diameter of a catheter engaged in the incomplete cylindrical bore such that the first and second elongate slots mechanically capture the catheter but may be easily slid along an axial length of the catheter;
wherein the engaged catheter may be gripped by the application of a compressive force to the respective compression regions so as to elastically deform the rib, reduce the gap between the distal ends of the first and second rigid plates and compress the outside surface of the catheter with respective inside surfaces of the first and second elongate slots; and
wherein the catheter may be engaged or disengaged from the elongate slots without deformation of the catheter by the application of a separation force to the respective separation regions so as to expand the gap between the distal ends until the gap is greater than the outside diameter of the catheter.

20. A method for gripping and releasing a catheter utilizing a catheter grip, comprising:
applying a first separation force to a separation region of a first rigid plate of a catheter grip and applying a second separation force which is opposed to the first separation force to a separation region of a second rigid plate of the catheter grip so as to elastically deform a resilient rib which pivotally couples the first rigid plate to the second rigid plate and to expand a gap between a distal end of the first rigid plate and a distal end of the second rigid plate;
engaging the catheter with a first elongate slot of the first rigid plate and with a second elongate slot of the second rigid plate;
applying a first compressive force to a compression region of the first rigid plate and a second compressive force which is opposed to the first compressive force to a compression region of the second rigid plate so as to elastically deform the resilient rib and to reduce the gap between the distal ends of the first and second rigid plates thereby engaging an outside surface of the catheter with respective inside surfaces of the first and second elongate slots; and
manipulating catheter with the catheter grip.

21. The method of claim 20 further comprising releasing the first and second compressive forces so as to transition the resilient rib to a relaxed state wherein the first and second elongate slots mechanically capture the outside surface of the catheter but no longer grip the outside surface of the catheter so as to allow the catheter grip to be slid along an axial length of the catheter.

22. The method of claim 21 further comprising sliding the catheter grip along the axial length of the catheter.

23. The method of claim 20 wherein elastically deforming the resilient rib further comprises pivoting the first rigid plate and the second rigid plate such that a first longitudinal axis of the first elongate slot and a second longitudinal axis of the second elongate slot remain aligned during the pivoting of the first and second rigid plates.

24. The method of claim 23 wherein pivoting the first rigid plate and the second rigid plate comprises pivoting the first rigid plate and the second rigid plate about a pivot axis which is disposed within the resilient rib, the resilient rib extending from an inside surface of the first rigid plate to an inside surface of the second rigid plate and the pivot axis being substantially parallel to the first and second longitudinal axes.

25. The method of claim 20 wherein applying the first and second separation forces to respective separation regions of the first and second rigid plates further comprises applying the first and second separation forces until the gap between the distal end of the first rigid plate and the distal end of the second rigid plate is greater than an outside diameter of the catheter.

26. The method of claim 20 wherein engaging the catheter with a first elongate slot of the first rigid plate and with a second elongate slot of the second rigid plate further comprises inserting the catheter without deformation of the catheter through the gap between the distal ends of the first and second rigid plates.

27. A method for gripping and releasing a catheter utilizing a catheter grip comprising:
providing a catheter grip comprising:
a first rigid plate including a proximal end, a distal end, a compression region on an outside surface of the first rigid plate, a separation region on the outside surface proximal of the compression region and a first elongate slot with a cylindrical contour extending transversely across an inside surface of the distal end, the first elongate slot including a first longitudinal axis of curvature;
a second rigid plate including a proximal end, a distal end, a compression region on an outside surface of the second rigid plate, a separation region on the outside surface of the second rigid plate proximal of the compression region and a second elongate slot with a cylindrical contour extending transversely across an inside surface of the distal end of the second rigid plate, the second elongate slot including a second longitudinal axis of curvature, the second elongate slot being disposed opposite to and in alignment with the first elongate slot;
a resilient rib which pivotally couples the first rigid plate to the second rigid plate such that respective longitudinal axes of the first elongate slot and second elongate slot remain aligned during pivoting of the first rigid plate and second rigid plate, which extends from the inside surface of the first rigid plate to the inside surface of the second rigid plate; which allows the first rigid plate and second rigid plate to be displaced in a relative pivoting motion along a pivot axis which is parallel to the respective longitudinal axes of the first and second elongate slots, and which is disposed between the respective separation region and compression region of each of the first and second rigid plates;
applying a separation force to the respective separation regions so as to expand a gap between the distal ends of the first and second plates until the gap is greater than an outside diameter of the catheter;
engaging the catheter with the elongate slots without deformation of the catheter by inserting the catheter through the gap between the distal ends of the first and second plates;
applying a compressive force to the respective compression regions so as to elastically deform the rib and reduce the gap between the distal ends of the first and second plates thereby engaging the outside surface of the catheter with the respective inside surfaces of the first and second elongate slots; and
releasing the compressive force thereby transitioning the resilient rib to a relaxed state wherein the first and second rigid plates are substantially parallel to each other and the first and second longitudinal axes of curvature of the respective elongate slots are aligned so as to define an incomplete cylindrical bore with a nominal inside diameter slightly larger than a nominal outer diameter of the catheter engaged in the incomplete cylindrical bore such that the first and second elongate slots mechanically capture the catheter but may be easily slid along an axial length of the catheter, and then sliding the catheter grip along the axial length of the catheter.
